(19) United States
(12) Patent Application Publication
(10) Pub. No.: US 2026/0216280 A1
(43) Pub. Date: Jul. 30, 2026

(54) COMPOSITION FOR PREVENTING AND TREATING ISCHEMIC CEREBROVASCULAR DISEASE COMPRISING GINTONIN

(71) Applicant: Dongguk University Industry-Academic Cooperation Foundation, Seoul (KR)

(72) Inventors: Dong-Eog KIM, Seoul (KR); Sun Hye CHOI, Seongnam-si (KR); Seung Yeol NAH, Seoul (KR); Won-kyung HONG, Goyang-si (KR)

(73) Assignee: Dongguk University Industry-Academic Cooperation Foundation, Seoul (KR)

(21) Appl. No.: 19/143,552

(22) PCT Filed: Dec. 18, 2023
(86) PCT No.: PCT/KR2023/020867

§ 371 Date: Jun. 26, 2025

(30) Foreign Application Priority Data
Dec. 27, 2022 (KR) 10-2022-0186469

(51) Int. Cl. A61K 38/16; A61K 9/00; A61P 9/10

(52) U.S. Cl. CPC A61K 38/168; A61K 9/0056; A61P 9/10

(57) ABSTRACT

The present invention relates to a composition for the prevention and treatment of ischemic cerebrovascular diseases, including cerebral infarction, comprising gintonin as an active ingredient. Gintonin of the present invention reduces cerebral infarction volume and can increase survival rate after the onset of cerebral infarction. In addition, the neuroprotective effect and damage prevention effect of gintonin pretreatment on human brain microvascular endothelial cells (HBMECs), which are human brain-derived vascular endothelial cells, and the neuroprotective effect and therapeutic effect of gintonin post-treatment were confirmed. As a result of mechanism verification, it was found that gintonin was effective in recovering the level of reduced nitric oxide (NO), which is known to be involved in the onset and pathophysiology of cerebral infarction. Therefore, the present invention can be used to improve the prevention and treatment of ischemic cerebrovascular diseases, including cerebral infarction, and thus, medical costs can be reduced.

### TECHNICAL FIELD

This application claims priority to and the benefit of Korean Patent Application No. 10-2022-0186469, filed on Dec. 27, 2022, and the disclosure of which is incorporated herein by reference in its entirety.

The present invention relates to a composition for the prevention and treatment of an ischemic cerebrovascular disease, such as cerebral infarction, including gintonin as an active ingredient. Gintonin according to the present invention has the effect of preventing cerebral infarction, reducing the volume of cerebral infarction, and increasing the survival rate after cerebral infarction, and thus, it can be advantageously used for the prevention and treatment of an ischemic cerebrovascular disease.

The present invention was completed with the support of the National Research Foundation of Korea under Project No. 2021R1A6A1A03038865 (Project Identification Number 1345347342).

### BACKGROUND ART

Cerebral infarction is an ischemic disease in which cerebral blood vessels are blocked and brain tissue dies. It is the most common type of stroke, is a major cause of cerebrovascular disease along with cerebral hemorrhage, and is known to be one of the most common causes of death worldwide.

According to the Global Burden of Disease report, as of 2019, stroke is a serious disease that is the third leading cause of death and disability worldwide, costing approximately $891 billion (approximately 1,100 trillion won).

According to the results of a study conducted on 11,136 patients with acute cerebral infarction hospitalized in 14 general hospitals nationwide from 2011 to 2013, the average medical expenses spent in the year before the onset of cerebral infarction was approximately 7.6 million won, but in the first year after the onset of cerebral infarction, it increased by about four times to approximately 33 million won. Additionally, it was found that the total medical costs incurred by a cerebral infarction patient over 5 years averages approximately 100 million won.

Additionally, in the case of patients who fully recovered without aftereffects 3 months after discharge, the total medical expenses spent over 5 years was approximately 47 million won, whereas in the case of patients who were unable to walk or perform daily activities without the help of others, it was confirmed that the total expenses spent were approximately 240 million won, which is nearly 5 times more. This suggests that not only the degree of recovery but also the economic burden of patients with cerebral infarction vary significantly depending on how they are treated in the acute phase. Therefore, the prevention and treatment of cerebral infarction are very important, even in terms of reducing national medical costs.

As the population ages and dietary patterns change, the incidence of stroke is increasing every year, and this is further increasing the global burden. Primary and secondary brain damage due to stroke causes inflammation, neuronal death, ischemia-reperfusion injury, blood-brain barrier damage, the release of neurotoxic substances, vitreous production, oxidative stress and cerebral edema through complex pathophysiological processes. Current ischemic stroke treatments mainly include thrombectomy, stents and angioplasty, surgical treatment (compression craniectomy and distal carotid artery resection), thrombolysis, and rehabilitation training. Through these, it can improve the prognosis and quality of life of stroke patients to some extent. In particular, it is true that the prognosis of stroke has been greatly improved with thrombolysis or mechanical thrombectomy, but even with such treatment, the majority of patients are unable to lead an independent life. Therefore, the development of new stroke treatments is necessary.

As mentioned above, the current standard treatment for acute cerebral infarction is administering thrombolytics within 4.5 hours of onset or performing thrombectomy within 24 hours of onset to recanalize blocked cerebral blood vessels. However, even after recanalization, many patients die or live with disabilities because brain cells die due to reperfusion damage, inflammation and delayed death. Therefore, in order to develop new treatments, methods to increase the efficacy of thrombolytics, methods to rebuild blood vessels, and methods to reduce inflammatory (secondary) brain damage are being studied.

Gintonin is a new glycolipoprotein extracted from ginseng and is a non-ginsenoside substance different from the well-known saponin. The main components of gintonin are lysophosphatidic acid (LPA) and other proteins, and LPA receptor (LPAR) is a specific target receptor for gintonin and has high affinity. In other words, gintonin is an LPAR ligand derived from ginseng. Previous studies have observed the beneficial role of gintonin in mouse models of chronic neurodegenerative diseases such as Alzheimer's disease and Parkinson's disease. In addition, research on the efficacy of gintonin is actively underway. However, research and verification related to cerebral infarction treatment have not yet been conducted.

Meanwhile, nitric oxide (NO) is a gas naturally produced in the body and is a very important signaling molecule that plays many different roles in bodily functions. One of the first important functions of nitric oxide (NO) is vasodilation, which plays an important role in maintaining healthy blood pressure and circulation by increasing blood flow by relaxing and widening blood vessels. In addition, nitric oxide performs immune functions by regulating the immune system and preventing infection, and is involved in various cell signaling pathways that regulate various cell signaling functions such as cell growth, differentiation and apoptosis. The main physiological functions of nitric oxide include maintaining vascular tone, reducing inflammatory responses, balancing thrombo-thrombotic homeostasis and regulating cell growth.

Nitric oxide also plays an important role in brain development and function, and it helps regulate blood flow to the brain, protects nerve cells from damage, and promotes learning and memory. Nitric oxide also helps maintain cardiovascular health by preventing blood clots and keeping blood vessels healthy, and is used to treat cardiovascular diseases such as erectile dysfunction.

There are three types of nitric oxide synthase (NOS). eNOS is the most common type of NOS, and it is found in endothelial cells arranged along blood vessels and plays a role in producing nitric oxide that causes vasodilation. iNOS is found in immune cells and is involved in inflammatory responses, while nNOS is found in nerve cells and is involved in learning and memory.

Nitric oxide (NO) is a very important molecule, but can be harmful if produced in excess. For example, the high levels of NO can cause damage to cells and tissues. This can occur in situations such as sepsis or inflammation.

Overall, nitric oxide is a versatile molecule that plays an important role in a variety of body functions and is important to keep in balance. As mentioned above, nitric oxide is a substance that plays an important role in dilating blood vessels and improving blood circulation, and when cerebral infarction occurs, the production of nitric oxide in brain tissue decreases. This can reduce cerebral blood flow and worsen brain damage.

Meanwhile, Korean Registered Patent No. 10-2011-0128734 discloses ‘gintonin that inhibits cancer metastasis and a composition for inhibiting cancer metastasis and cancer metastasis containing the gintonin as an active ingredient’, and also Korean Registered Patent No. 10-1077226-0000 discloses ‘a composition for preventing and treating a degenerative cranial nervous system disease including gintonin as an active ingredient’, but as in the present invention, there has been no report on a composition including gintonin as an active ingredient for preventing and treating cerebral infarction or ischemic cerebral infarction.

### DISCLOSURE

### Technical Problem

While researching substances that are highly effective and easily accessible to patients for the prevention and treatment of ischemic cerebrovascular diseases, including cerebral infarction, as described above, the present invention was completed by confirming the therapeutic effect of gintonin on a mouse model of local ischemic stroke.

Therefore, an object of the present invention is to provide a composition containing gintonin as an active ingredient for the prevention and treatment of ischemic cerebrovascular diseases, including cerebral infarction.

### Technical Solution

The present invention provides a pharmaceutical composition for preventing or treating an ischemic cerebrovascular disease, including gintonin.

According to a preferred embodiment of the present invention, the gintonin is lysophosphatidic acids in gintonin.

According to a preferred embodiment of the present invention, the ischemic cerebrovascular disease is ischemic cerebral infarction.

According to a preferred embodiment of the present invention, the ischemic cerebrovascular disease is caused by decreased nitric oxide production.

According to a preferred embodiment of the present invention, the composition reduces a volume of cerebral infarction.

According to a preferred embodiment of the present invention, the composition increases a survival rate of a subject with cerebral infarction.

In addition, the present invention provides a health functional food composition for preventing or ameliorating an ischemic cerebrovascular disease, including gintonin.

In addition, the present invention provides a composition for protecting a nerve cell, including gintonin.

According to a preferred embodiment of the present invention, the gintonin is lysophosphatidic acids in gintonin.

According to a preferred embodiment of the present invention, the nerve cell is a nerve cell damaged due to decreased nitric oxide production.

According to a preferred embodiment of the present invention, the nerve cell is a nerve cell derived from cerebral blood vessels.

In addition, the present invention provides a health functional food composition for protecting a nerve cell, including gintonin.

In addition, the present invention provides a method for treating an ischemic cerebrovascular disease, including administering a composition including gintonin to a patient with an ischemic cerebrovascular disease.

In addition, the present invention provides the use of gintonin for use in the treatment of an ischemic cerebrovascular disease.

In addition, the present invention provides the use of gintonin for the manufacture of a therapeutic agent for an ischemic cerebrovascular disease.

‘Prevention’ of the present invention refers to all actions that suppress or delay the onset of an ischemic cerebrovascular disease or related diseases due to the gintonin of the present invention.

‘Amelioration’ or ‘treatment’ of the present invention means any action that improves or benefits parameters that are related to an ischemic cerebrovascular disease or related diseases, such as the degree of symptoms, due to the gintonin of the present invention.

The present invention relates to the effects of gintonin in preventing, ameliorating or treating an ischemic cerebrovascular disease, including cerebral infarction, and protecting nerve cells. The gintonin may mean a substance isolated from white ginseng, red ginseng or red ginseng peel.

The inventors of the present invention verified that gintonin has a neuroprotective effect in a mouse model of focal ischemic stroke as follows. First of all, C57BL/6N mice were orally administered with saline (n=50) and 150 mg/kg (n=65) or 300 mg/kg (n=20) of gintonin once a day for 1 week. Additionally, 15 randomly selected mice among the animals in the 150 mg/kg group were injected intraperitoneally with an LPAR antagonist (Ki16425, 30 mg/kg) before the oral administration of gintonin. Cerebral autoregulatory dysfunction (CAD) was induced in all mice through the intraperitoneal administration of a NOS antagonist, and then, hemispheric ischemia was induced through common carotid artery occlusion (CCAO). Survival rate and final infarct volume after 1 week were compared between groups (Log-rank test and Student t-test, respectively). 150 mg/kg of gintonin (vs. saline) increased a 1-week survival rate (35/50 [70 %] vs. 23/50 [46 %], p=0.009) and the size of infarct (92.20±14.53 mm³ vs. 138.70±16 mm³, p=0.005) was reduced. 300 mg/kg of gintonin tended to increase the survival rate (12/20 [60%], p=0.18 vs. saline) to a statistically non-significant level and infarct size (40±36 mm³, p=0.75 vs. saline) did not significantly decrease or increase (FIG. 1). Pretreatment with an LPA receptor antagonist inhibited the effects of 150 mg/kg of gintonin on the reduction in survival rate (6/15 [40%], p=0.61 vs. saline) and infarct size (183.81±31.97mm³, p=0.88 vs. saline) after stroke (FIG. 2). In conclusion, 150 mg/kg of gintonin increased a survival rate and decreased an infarct volume after stroke, which is presumed to be due to LPAR inhibition.

Based on the results of this in vivo animal model verification, the following mechanism was verified regarding the relationship between nitric oxide and gintonin in preventing, ameliorating or treating cerebral infarction through cell experiments using HBMEC (Human Brain Microvascular Endothelial Cell). It was confirmed that the decreased nitric oxide level was restored by gintonin by treatment with a nitric oxide synthase (NOS) inhibitor (L-NAME) (FIGS. 3 to 5). In addition, the result of failure to recover the decreased nitric oxide level caused by gintonin (10 ug/mL) after pretreatment with the LPAR antagonist Ki16425 (5 uM) was consistent with the results of the in vivo animal model experiment.

Accordingly, the present invention may provide a pharmaceutical composition for preventing or treating an ischemic cerebrovascular disease, including gintonin.

According to a preferred embodiment of the present invention, the gintonin may be lysophosphatidic acids in gintonin.

According to a preferred embodiment of the present invention, the ischemic cerebrovascular disease may be ischemic cerebral infarction.

According to a preferred embodiment of the present invention, the ischemic cerebrovascular disease may be caused by decreased nitric oxide production.

According to a preferred embodiment of the present invention, the composition may reduce a volume of cerebral infarction.

According to a preferred embodiment of the present invention, the composition may increase a survival rate of a subject with cerebral infarction.

The pharmaceutical composition of the present invention may be in various oral or parenteral preparations. When the composition is formulated, the composition may be prepared using one or more buffers (e.g., saline or PBS), antidiabetic agents, bacteriostats, chelating agents (e.g., EDTA or glutathione), fillers, extenders, binders, adjuvants (e.g., aluminum hydroxide), suspensions, thickeners, wetting agents, disintegrants or surfactants, diluents or excipients.

Solid preparations for oral administration include tablets, pills, powders, granules, capsules and the like, and these solid preparations are prepared by mixing one or more compounds with at least one excipient, for example, starch (including corn starch, wheat starch, rice starch, potato starch, etc.), calcium carbonate, sucrose, lactose, dextrose, sorbitol, mannitol, xylitol, erythritol maltitol, cellulose, methyl cellulose, sodium carboxymethylcellulose, and hydroxypropylmethyl-cellulose or gelatin. For example, tablets or sugar-coated tablets may be obtained by mixing an active ingredient with a solid excipient, grinding the mixture, adding a suitable auxiliary agent, and then processing the mixture into a granule mixture.

Additionally, in addition to simple excipients, lubricants such as magnesium stearate, talc and the like are also used. Liquid preparations for oral administration correspond to suspensions, oral solutions, emulsions or syrups. In addition to the commonly used simple diluents such as water and liquid paraffin, they may include various excipients such as wetting agents, sweeteners, fragrances or preservatives. Additionally, in some cases, cross-linked polyvinylpyrrolidone, agar, alginic acid or sodium alginate may be added as a disintegrant, and anti-coagulants, flavoring agents, emulsifiers, solubilizers, dispersants, flavoring agents, antioxidants, and packaging agents., pigments and preservatives may be additionally included.

Preparations for parenteral administration include sterilized aqueous solutions, non-aqueous solutions, suspensions, emulsions, freeze-dried preparations or suppositories. Non-aqueous solvents and suspensions may include propylene glycol, polyethylene glycol, vegetable oil such as olive oil, and injectable ester such as ethyl oleate. As a base for suppositories, witepsol, macrogol, tween 61, cacao, laurel, glycerol, gelatin and the like may be used.

The composition of the present invention may be administered orally or parenterally, and when administered parenterally, it may be formulated according to methods known in the art in the form of an injection for intraperitoneal, intravenous, intramuscular, subcutaneous or intracerebrovascular injection.

The injection must be sterilized and protected from contamination by microorganisms such as bacteria and fungi. Examples of a suitable carrier for injection may be, but are not limited to, a solvent or a dispersion medium including water, ethanol, polyols (e.g., glycerol, propylene glycol, liquid polyethylene glycol, etc.), mixtures thereof and/or vegetable oils. More preferably, as a suitable carrier, it is possible to use an isotonic solution such as Hank's solution, Ringer's solution, triethanolamine-containing phosphate buffered saline (PBS) or sterile water for injection, 10% ethanol, 40% propylene glycol and 5% dextrose. In order to protect the injection from microbial contamination, various antimicrobial agents and antifungal agents such as paraben, chlorobutanol, phenol, sorbic acid and thimerosal may be additionally included. Additionally, in most cases, the injection may additionally include an isotonic agent such as sugar or sodium chloride.

The composition of the present invention is administered in a pharmaceutically effective amount, and the pharmaceutically effective amount refers to an amount that is sufficient to treat a disease at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, factors including simultaneously used drugs and other factors well known in the medical field. The composition of the present invention may be administered as an individual therapeutic agent or in combination with other therapeutic agents, may be administered sequentially or simultaneously with therapeutic agents in the related art, and may be administered in a single dose or multiple doses. That is, the total effective amount of the composition of the present invention may be administered to a patient in a single dose or may be administered by a fractionated treatment protocol, in which multiple doses are administered over a long period of time. It is important to administer the composition in a minimum amount that can obtain the maximum effect without any side effects, in consideration of all the aforementioned factors, and this amount may be easily determined by the person skilled in the art.

The dosage of the pharmaceutical composition of the present invention may vary depending on the patient's weight, age, gender, health condition, diet, administration time, administration method, excretion rate and the severity of disease.

The composition of the present invention may be used alone or in combination with surgery, radiation therapy, hormone therapy, chemotherapy and methods using biological response modifiers.

In addition, the present invention may provide a health functional food composition for preventing or ameliorating an ischemic cerebrovascular disease, including gintonin.

According to a preferred embodiment of the present invention, the gintonin may be lysophosphatidic acids in gintonin.

According to a preferred embodiment of the present invention, the ischemic cerebrovascular disease may be ischemic cerebral infarction.

According to a preferred embodiment of the present invention, the ischemic cerebrovascular disease may be caused by decreased nitric oxide production.

According to a preferred embodiment of the present invention, the composition may reduce a volume of cerebral infarction.

According to a preferred embodiment of the present invention, the composition may increase a survival rate of a subject with cerebral infarction.

The food composition according to the present invention may be prepared in various forms according to conventional methods known in the art. General food may be prepared by adding the gintonin of the present invention to a beverage (including an alcoholic beverage), fruit and a processed food thereof (e.g., canned fruit, bottled food, jam, marmalade, etc.), fish, meat and processed food thereof (e.g., ham, sausage, corned beef, etc.), bread and noodles (e.g., thick wheat noodles, buckwheat noodles, instant noodles, spaghetti, macaroni, etc.), fruit juices, various drinks, cookies, Korean hard taffy, dairy products (e.g., butter, cheese, etc.), edible vegetable oils, margarine, vegetable protein, retort foods, frozen food and various seasonings (e.g., soybean paste, soy sauce, sauce, etc.) and the like, but is not limited thereto. In addition, the gintonin of the present invention may be manufactured by adding the same to capsules, tablets, pills and the like, but is not limited thereto as a nutritional supplement. In addition, as health functional food, it is not limited to this, but for example, the gintonin of the present invention itself may be manufactured in the form of tea, juice and drink, and consumed (health beverage) by liquefying, granulating, encapsulating and powdering. Additionally, in order to use the gintonin of the present invention in the form of a food additive, it may be prepared and used in the form of powder or concentrate. In addition, the gintonin of the present invention may be prepared in the form of a composition by mixing the same with a known active ingredient that is known to have an effect of preventing or ameliorating an ischemic cerebrovascular disease.

When the gintonin of the present invention is used as a health drink, the health drink composition may contain various flavoring agents or natural carbohydrates as additional ingredients like ordinary drinks. The above-mentioned natural carbohydrates may be monosaccharides such as glucose and fructose; disaccharides such as maltose and sucrose; polysaccharides such as dextrins and cyclodextrins; and sugar alcohols such as xylitol, sorbitol, or erythritol. For sweeteners, natural sweeteners such as thaumatin and stevia extract; synthetic sweeteners such as saccharin and aspartame and the like may be used. The proportion of the natural carbohydrate is generally about 0.01 to 0.04 g, and preferably, about 0.02 to 0.03 g, per 100 mL of the composition of the present invention.

In addition, the gintonin of the present invention may be contained as an active ingredient in a health functional food composition for preventing or ameliorating an ischemic cerebrovascular disease, but the amount is not specifically limited to an amount that is effective for achieving the effect of preventing or ameliorating an ischemic cerebrovascular disease, and it is preferably 0.01 to 100% by weight based on the total weight of the entire composition. The health functional food composition of the present invention may be prepared by mixing gintonin with other active ingredients that are known to be effective in an ischemic cerebrovascular disease.

In addition to the above, the health functional food of the present invention may contain various nutrients, vitamins, electrolytes, flavors, colorants, pectic acid, salts of pectic acid, alginic acid, salts of alginic acid, organic acids, protective colloidal thickeners, pH adjusters, stabilizers, preservatives, glycerin, alcohol or carbonating agent. In addition, the health functional food of the present invention may contain pulp for the production of natural fruit juice, fruit juice beverage or vegetable beverage. These ingredients may be used independently or in combination.

In addition, the present invention may provide a composition for protecting a nerve cell, including gintonin.

According to a preferred embodiment of the present invention, the gintonin may be lysophosphatidic acids in gintonin.

According to a preferred embodiment of the present invention, the nerve cell may be a nerve cell that is damaged due to reduced nitric oxide production.

According to a preferred embodiment of the present invention, the nerve cell may be a nerve cell that is derived from cerebral blood vessels.

In addition, the present invention may provide a health functional food composition for protecting a nerve cell, including gintonin.

According to a preferred embodiment of the present invention, the gintonin may be lysophosphatidic acids in gintonin.

According to a preferred embodiment of the present invention, the nerve cell may be a nerve cell that is damaged due to reduced nitric oxide production.

According to a preferred embodiment of the present invention, the nerve cell may be a nerve cell that is derived from cerebral blood vessels.

The food composition according to the present invention may be prepared in various forms according to conventional methods known in the art. General food may be prepared by adding the gintonin of the present invention to a beverage (including an alcoholic beverage), fruit and a processed food thereof (e.g., canned fruit, bottled food, jam, marmalade, etc.), fish, meat and processed food thereof (e.g., ham, sausage, corned beef, etc.), bread and noodles (e.g., thick wheat noodles, buckwheat noodles, instant noodles, spaghetti, macaroni, etc.), fruit juices, various drinks, cookies, Korean hard taffy, dairy products (e.g., butter, cheese, etc.), edible vegetable oils, margarine, vegetable protein, retort foods, frozen food and various seasonings (e.g., soybean paste, soy sauce, sauce, etc.), but is not limited thereto. In addition, the gintonin of the present invention may be manufactured by adding the same to capsules, tablets, pills and the like, but is not limited thereto as a nutritional supplement. In addition, as health functional food, it is not limited to this, but for example, the gintonin of the present invention itself may be manufactured in the form of tea, juice and drink, and consumed (health beverage) by liquefying, granulating, encapsulating and powdering. Additionally, in order to use the gintonin of the present invention in the form of a food additive, it may be prepared and used in the form of powder or concentrate. In addition, the gintonin of the present invention may be prepared in the form of a composition by mixing the same with a known active ingredient that is known to have an effect of preventing or ameliorating an ischemic cerebrovascular disease.

When the gintonin of the present invention is used as a health drink, the health drink composition may contain various flavoring agents or natural carbohydrates as additional ingredients like ordinary drinks. The above-mentioned natural carbohydrates may be monosaccharides such as glucose and fructose; disaccharides such as maltose and sucrose; polysaccharides such as dextrins and cyclodextrins; and sugar alcohols such as xylitol, sorbitol and erythritol. For sweeteners, natural sweeteners such as thaumatin and stevia extract; and synthetic sweeteners such as saccharin and aspartame may be used. The proportion of the natural carbohydrate is generally about 0.01 to 0.04 g, and preferably, about 0.02 to 0.03 g, per 100 ml of the composition of the present invention.

In addition, the gintonin of the present invention may be contained as an active ingredient in a health functional food composition for protecting a nerve cell, but the amount is not specifically limited to an amount that is effective for achieving the effect of protecting a nerve cell, and it is preferably 0.01 to 100% by weight based on the total weight of the entire composition. The health functional food composition of the present invention may be prepared by mixing gintonin with other active ingredients that are known to be effective in an ischemic cerebrovascular disease.

In addition to the above, the health functional food of the present invention may contain various nutrients, vitamins, electrolytes, flavors, colorants, pectic acid, salts of pectic acid, alginic acid, salts of alginic acid, organic acids, protective colloidal thickeners, pH adjusters, stabilizers, preservatives, glycerin, alcohol or carbonating agent. In addition, the health functional food of the present invention may contain pulp for the production of natural fruit juice, fruit juice beverage or vegetable beverage. These ingredients may be used independently or in combination.

In addition, the present invention may provide a method for treating an ischemic cerebrovascular disease, including administering a composition including gintonin to a patient with an ischemic cerebrovascular disease.

According to a preferred embodiment of the present invention, the gintonin may be lysophosphatidic acids in gintonin.

According to a preferred embodiment of the present invention, the ischemic cerebrovascular disease may be ischemic cerebral infarction.

According to a preferred embodiment of the present invention, the ischemic cerebrovascular disease may be caused by decreased nitric oxide production.

According to a preferred embodiment of the present invention, the treatment method may be to reduce a volume of cerebral infarction.

According to a preferred embodiment of the present invention, the treatment method may increase a survival rate of a subject with cerebral infarction.

In addition, the present invention may provide the use of gintonin for use in the treatment of an ischemic cerebrovascular disease.

According to a preferred embodiment of the present invention, the ischemic cerebrovascular disease may be ischemic cerebral infarction.

According to a preferred embodiment of the present invention, the ischemic cerebrovascular disease may be caused by decreased nitric oxide production.

According to a preferred embodiment of the present invention, the treatment may be to reduce a volume of cerebral infarction.

According to a preferred embodiment of the present invention, the treatment may increase a survival rate of a subject with cerebral infarction.

According to a preferred embodiment of the present invention, the gintonin may be lysophosphatidic acids in gintonin.

In addition, the present invention may provide the use of gintonin for use in the manufacture of a therapeutic agent for an ischemic cerebrovascular disease.

According to a preferred embodiment of the present invention, the ischemic cerebrovascular disease may be ischemic cerebral infarction.

According to a preferred embodiment of the present invention, the ischemic cerebrovascular disease may be caused by decreased nitric oxide production.

According to a preferred embodiment of the present invention, the therapeutic agent may reduce a volume of cerebral infarction.

According to a preferred embodiment of the present invention, the therapeutic agent may increase a survival rate of a subject with cerebral infarction.

According to a preferred embodiment of the present invention, the gintonin may be lysophosphatidic acids in gintonin.

### Advantageous Effects

Gintonin, which includes an LPAR ligand-lysophosphatidic acid receptor (LPAR) derived from ginseng according to the present invention, increased the post-stroke survival rate and reduced the infarct volume in mice in a mouse model of focal ischemic stroke. Additionally, in cell experiments based on human brain microvascular endothelial cells (HBMEC), it was confirmed that the decreased nitric oxide (NO) level was restored by treatment with an inhibitor (NOSi) of nitric oxide synthase (NOS).

Therefore, gintonin has the effect of being advantageously used for the prevention and treatment of ischemic cerebrovascular diseases, including cerebral infarction. As a result, it is expected to provide market competitiveness by reducing medical costs for treatment and the like.

### DESCRIPTION OF DRAWINGS

FIG. 1 shows the results showing the effect on a survival curve by evaluating a survival rate after cerebral infarction induction modeling (CCAO+NOSi-mediated) by gintonin.

FIG. 2 shows the image results of mouse brains extracted and stained with 2,3,5-triphenyltetrazolium chloride (TTC) to investigate the effect of gintonin (GEF) treatment on the volume of cerebral infarct. White indicates the cerebral infarction area, and red indicates the undamaged area.

FIG. 3 is a graph showing the statistical comparison of the volume of cerebral infarction caused by ischemic stroke from the image results of extracting mouse brains and staining the same with 2,3,5-triphenyltetrazolium chloride (TTC) to investigate the effect of gintonin (GEF) treatment on the volume of cerebral infarction.

FIG. 4 shows the results of investigating the changes in nitric oxide (NO) production in human brain microvascular endothelial cells (HBMEC) and nitric oxide synthase (NOS) inhibitor treatment (100, 300 uM) that decreased nitric oxide production and restored nitric oxide production by gintonin pretreatment.

FIG. 5 shows the results of reanalysis by treatment with 100 uM nitric oxide synthase (NOS) inhibitor and pretreatment with gintonin. A of FIG. 5 shows the nitric oxide (NO) production of human brain microvascular endothelial cells (HBMEC) themselves, and B of FIG. 5 shows the results of analyzing the nitric oxide production converted into relative production by treatment conditions, which shows that the nitric oxide level decreased by treatment with 100 uM NOS inhibitor was restored by pretreatment with gintonin (F=9.819, P value=0.0019, P value summary=**, Significant diff. among means (P<0.05)=Yes, R squared=0.5670).

FIG. 6 shows the results of reanalysis of the results of treatment with 300 uM nitric oxide synthase (NOS) inhibitor and pretreatment with gintonin. Even in the case of treatment with 300 uM of a nitric oxide synthase inhibitor, it was confirmed that the decreased nitric oxide (NO) level was recovered by gintonin pretreatment.

FIG. 7 shows the results confirming that nitric oxide (NO) reduced by 100 and 300 uM of a nitric oxide synthase (NOS) inhibitor was recovered by post-treatment with gintonin. FIG. 7 A shows the results of nitric oxide production by human brain microvascular endothelial cells (HBMEC) according to treatment conditions, nitric oxide reduced by a synthesis inhibitor treatment (100, 300 uM), and nitric oxide recovered by post-treatment with gintonin; and FIG. 7 B shows the results of analyzing the relative nitric oxide production according to treatment conditions, confirming that the nitric oxide level reduced after a synthesis inhibitor treatment (100, 300 uM) was also recovered by post-treatment with gintonin.

FIG. 8 shows the results of investigating the effect on changes in nitric oxide (NO) production measured after pretreatment and culturing with an LPAR antagonist (Ki16425, 5 uM) in immortalized human brain microvascular endothelial cells (imHBMEC) for 30 minutes, treatment with gintonin at 10 ug/mL as in Example 3, and incubation for 1 hour, followed by treatment with 100 uM of a synthase inhibitor, and incubation for another hour, in order to determine whether the neuroprotective and damage prevention effects of gintonin 10 ug/mL treatment after pretreatment with a nitric oxide synthase (NOS) inhibitor were due to lysophosphatidic acid receptor (LPAR), which is a functional component of gintonin. It was confirmed that the decreased nitric oxide level by nitric oxide synthase inhibitor treatment was not recovered by gintonin when pretreated with the LPAR antagonist (Ki16425, 5 uM) for 30 minutes (A: F=6.474, P value=0.0023, P value summary =**, Significant diff. among means(P<0.05)=Yes, R squared=0.4473; B: F=13.89, P value =<0.0001, P value summary =****, Significant diff. among means(P<0.05)=Yes, R squared=0.6345).

### MODES OF THE INVENTION

Hereinafter, the present invention will be described in more detail through examples. These examples are only for illustrating the present invention, and it will be apparent to those skilled in the art that the scope of the present invention should not be construed as limited by these examples.

### Example 1

### Preparation of Focal Ischemic Stroke Mouse Model

In this experiment, C57Bl/6 male mice (25-28 g), which are most widely used worldwide for animal experiments on cerebral infarction, were used (DBL, Eumseong, Republic of Korea). Ten-week-old mice were maintained for at least one week prior to the experiment and housed in a cage with a 12-hour light/dark cycle at a controlled temperature (22° C.±1° C.) and humidity (50%±10%). Free access to food and water was allowed. All experiments were approved by the Animal Experiment Management Committee of Dongguk University Ilsan Hospital.

C57BL/6N mice were orally administered with saline (n=50) and 150 mg/kg (n=65) or 300 mg/kg (n=20) of gintonin once daily for 1 week. Additionally, 15 randomly selected mice among the animals in the 150 mg/kg group were injected intraperitoneally with an LPAR antagonist (Ki16425, 30 mg/kg) before oral administration of gintonin. After cerebral autoregulatory dysfunction (CAD) was induced in all mice through a one-time intraperitoneal administration of 100 mg/kg of NOS antagonist (L-NAME: N omega-Nitro-L-arginine methyl ester hydrochloride), a CCAO+NOSi-mediated focal ischemic stroke (cerebral infarction) mouse model was produced by inducing hemispheric ischemia through common carotid artery occlusion (CCAO).

### Example 2

### Evaluation of Survival Rate After Cerebral Infarction Modeling in Mice by Gintonin

The inventors of the present invention sought to evaluate a survival rate after cerebral infarction-induced modeling (CCAO+NOSi mediated) by gintonin.

Specifically, the survival rates of the saline-treated group (Con) and the gintonin-treated group (150 mg/kg GEF, 300 mg/kg GEF) prepared in <Example 1> were evaluated for 7 days. In order to obtain the average survival rate, the Kaplan-Meier method and log rank test were used.

As a result, the survival of the gintonin-treated animal group (150 mg/kg) was significantly increased (p=0.009) compared to the saline-treated group (p<0.05).

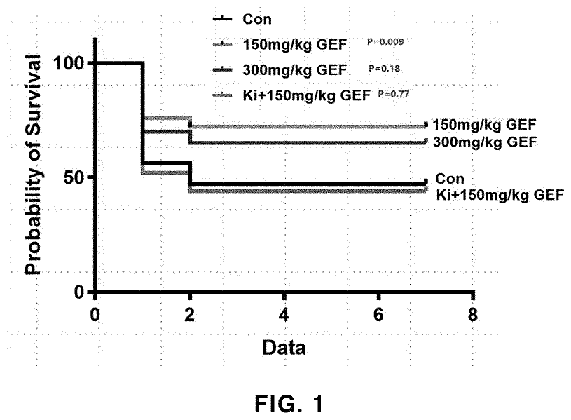

Additionally, in order to interfere with the receptor for lysophosphatidic acids, which is a substance contained in gintonin, when an LPA1/3 receptor antagonist (Ki16425) was pretreated and then gintonin was treated (Ki+150 mg/kg), the survival increase effect of gintonin was not observed. As a result of the mechanistic study, it appears that lysophosphatidic acid, which is a substance included in gintonin, prevents cerebral infarction by stimulating nitric oxide synthase (NOS) activation, thereby suppressing CCAO+NOSi-mediated cerebral infarction induction (FIG. 1).

### Example 3

### Inhibition of CCAO+NOSi-Mediated Cerebral Infarction Induction in Mice by Gintonin: Cerebral Infarction Prevention Effect

The inventors of the present invention sought to determine the effect of preventing cerebral infarction by measuring the cerebral infarct area after modeling (CCAO+NOSi) the induction of cerebral infarction by gintonin.

Specifically, the brains of the mouse models prepared in <Example 1> above were extracted, and 2 mm-thick brain tissue sections were made using a brain matrix, stained with 2% 2,3,5-triphenyltetrazolium chloride (TTC), and photographed with a digital camera. The area of cerebral infarction was measured from each section by using Image J software (the volume of cerebral infarction was calculated by multiplying the area by the thickness).

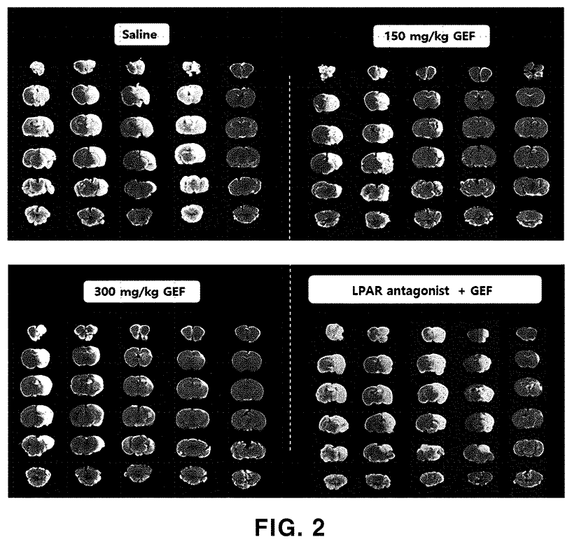

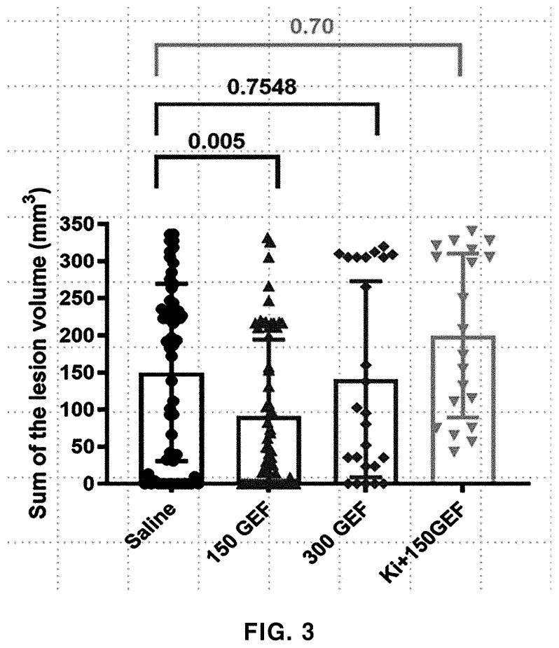

FIG. 2 is an image of the TTC staining result, and FIG. 3 is the result of a statistical comparison of cerebral infarction volumes calculated from the image. In FIG. 2, white indicates the cerebral infarction area, and red indicates the undamaged area. In FIG. 3, when comparing the saline group (Saline, 138.70±16 mm³) and the 150 mg/kg gintonin-treated group (150 mg/kg GEF, 92.20±14.53 mm³), it was confirmed that in the gintonin-treated group, the volume of cerebral infarction was significantly (about 33.57%) reduced (p=0.005). When the LPA1/3 receptor antagonist (Ki16425) was pretreated to inhibit the receptor action of lysophosphatidic acids, which are a substance contained in gintonin, and then gintonin was treated (LPAR antagonist +GEF, Ki+150GEF), the gintonin-induced cerebral infarction prevention effect was not observed. Based on these results, it is expected that gintonin has preventive and therapeutic effects on cerebral infarction by stimulating nitric oxide synthase (NOS) activation by the lysophosphatidic acid component (FIGS. 2 and 3).

### Example 4

### Neuroprotective and Damage Prevention Effects in Human Brain-Derived Vascular Endothelial Cells (HBMEC) by Gintonin

Based on the in vivo effects of gintonin in <Example 2> and <Example 3> above, the inventors of the present invention sought to determine whether gintonin also has effects in cell experiments (in vitro).

First of all, HBMEC (Human Brain Microvascular Endothelial Cell) or imHBMEC (immortalized Human Brain Microvascular Endothelial Cell) were cultured in an appropriate culture medium under air conditions of 37° C., 5% carbon dioxide and 95% relative humidity. When the cells reached 80 to 90% confluency (average ~2 days), 10 ug/mL of gintonin was first pretreated and incubated for 1 hour, and then, a nitric oxide synthase (NOS) inhibitor was treated and incubated for another hour, then, the effect of changes in nitric oxide (NO) production was investigated. Afterwards, the concentration of nitric oxide synthase inhibitor was treated at 100 and 300 uM.

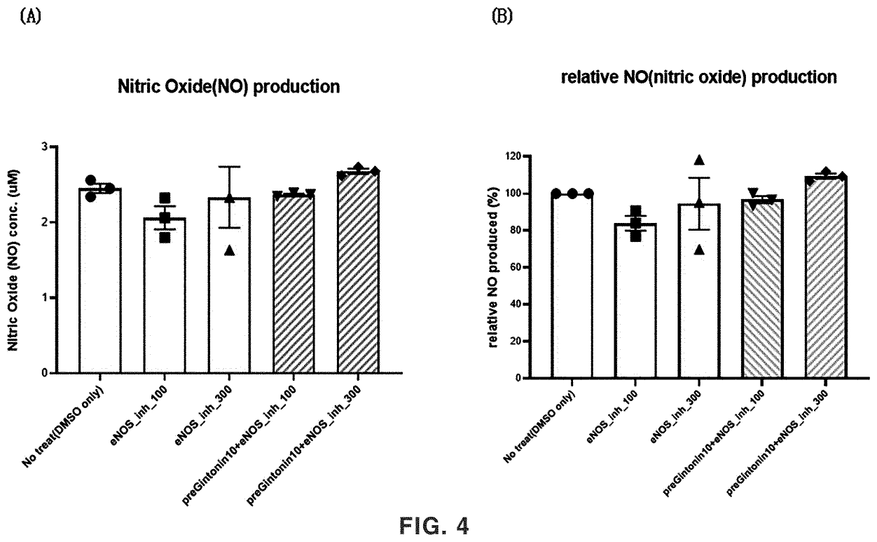

As a result, it was confirmed that nitric oxide reduced by 100 and 300 uM of the synthase inhibitor was recovered by pretreatment with gintonin (FIG. 4). FIG. 4A shows the production of nitric oxide produced in human brain-derived vascular endothelial cells HBMEC according to treatment conditions (only DMSO solvent treatment, no treat (DMSO only) to equalize the effect of the solvent of the treated reagents), nitric oxide reduced by synthase inhibitor treatment (100, 300 uM) (eNOS_inh_100, eNOS_inh_300), and nitric oxide recovered by pretreatment with gintonin (preGintonin10+eNOS_inh_100, preGintonin10+eNOS_inh_300). B of FIG. 4 shows the results of analyzing the amount of nitric oxide produced by treatment conditions by converting the same into relative production volume, taking into account the characteristics of cells that may slightly differ depending on conditions such as passage or confluency.

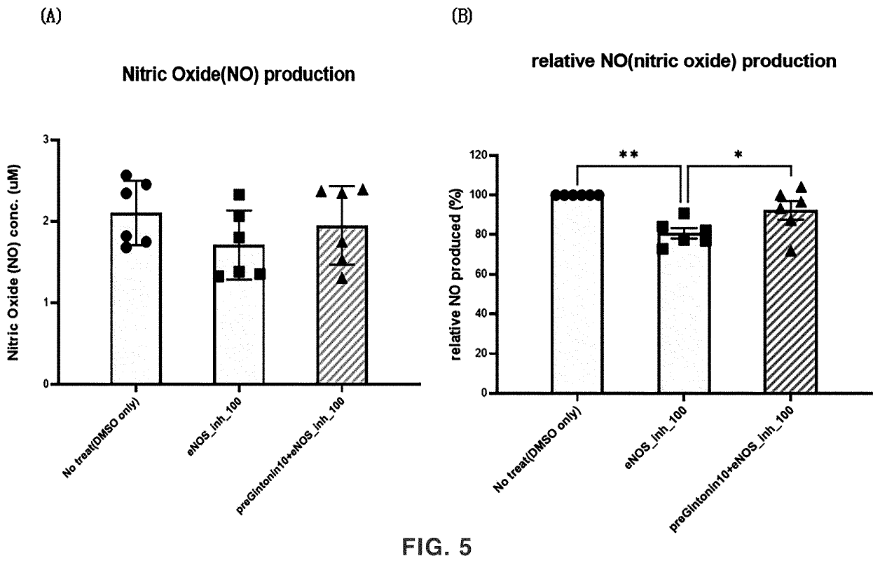

FIG. 5 is a diagram that reanalyzes only the results by treatment with 100 uM synthase inhibitor and pretreatment with gintonin according to treatment conditions. A of FIG. 5 shows the nitric oxide production of human brain-derived endothelial cells (HBMEC) themselves according to treatment conditions, and B of FIG. 5 shows the results of analyzing the nitric oxide production converted into the relative production amount according to treatment conditions. In the case of B of FIG. 5, the result of reducing nitric oxide by about 20% due to the synthase inhibitor was recovered by about 10 to 12% by pretreatment with gintonin, which was close to ~90% compared to the DMSO solvent treatment group (No treat (DMSO only)). Thus, it was confirmed that gintonin may have a neuroprotective or preventive effect by recovering the decreased nitric oxide level by treatment with a nitric oxide synthase inhibitor in human brain-derived endothelial cells HBMEC. In addition, the p value of the ANOVA statistical analysis result was significant at 0.0019.

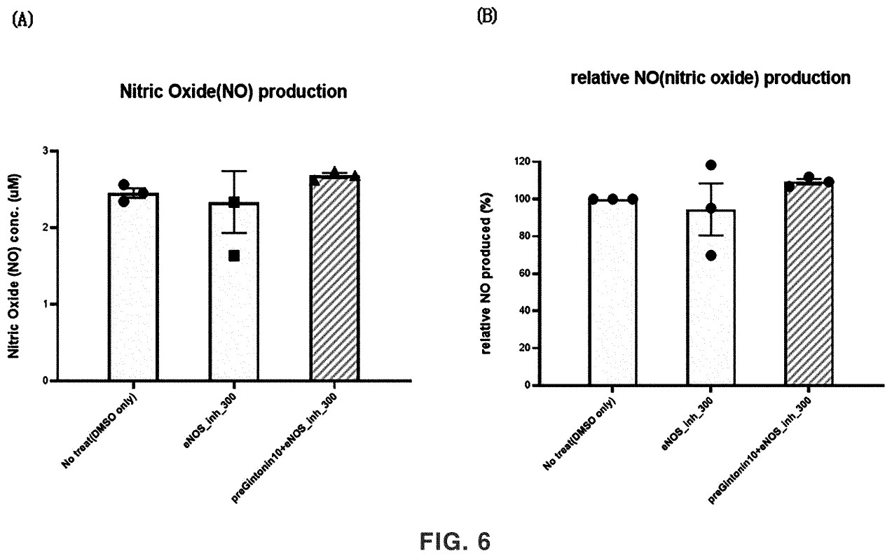

FIG. 6 is a diagram reanalyzing the results of treatment with 300 uM synthetase inhibitor and pretreatment with gintonin. A in FIG. 6 is the nitric oxide production of human brain-derived vascular endothelial cells (HBMEC) themselves by treatment condition, and B in FIG. 6 is the result of analyzing the nitric oxide production converted into relative production by treatment condition. £ It was confirmed that even in the case of treatment with 300 uM of the nitric oxide synthase inhibitor, the decreased nitric oxide level was recovered by gintonin pretreatment.

### Example 5

### Neuroprotective and Therapeutic Effects of Gintonin on Human Brain-Derived Vascular Endothelial Cells (HBMEC)

The inventors of the present invention sought to determine the neuroprotective and therapeutic effects of gintonin.

In the same manner as <Example 3> above, HBMEC (Human Brain Microvascular Endothelial Cell) or imHBMEC (immortalized Human Brain Microvascular Endothelial Cell) were cultured in an appropriate culture medium under air conditions of 37° C., 5% carbon dioxide and 95% relative humidity, and when the cells reached 80 to 90% confluency (average ~2 days), the cells were first pretreated with a nitric oxide synthase (NOS) inhibitor and incubated for 1 hour, followed by treatment with 10 ug/mL of gintonin for 1 hour. After further culturing, the effect on nitric oxide (NO) production was investigated to determine the neuroprotective and therapeutic effects of gintonin.

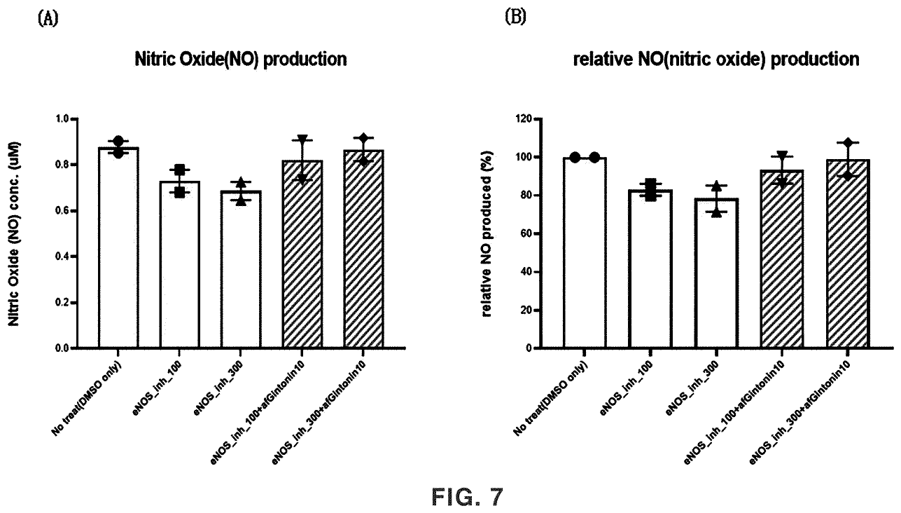

As a result, it was confirmed that nitric oxide reduced by 100 and 300 uM of the synthase inhibitor was recovered by post-treatment with gintonin (FIG. 7). FIG. 7 A shows the nitric oxide production (no treat (DMSO only)) produced by human brain-derived vascular endothelial cells (HBMEC) by treatment conditions and the nitric oxide reduced by synthase inhibitor treatment (100, 300 uM) (eNOS_inh_100, eNOS_inh_300)., and shows the results of nitric oxide (preGintonin10+eNOS_inh_100, preGintonin10+eNOS_inh_300) recovered by gintonin post-treatment. FIG. 7 B shows the results of analyzing the relative nitric oxide production by treatment condition.

As shown in the results of FIG. 7, it was confirmed that the decreased nitric oxide level after treatment with the synthase inhibitor (100, 300 uM) was recovered by post-treatment with gintonin. This confirmed that gintonin may have neuroprotective effects, damage prevention effects and therapeutic effects by restoring nitric oxide levels.

### Example 6

### Offset of Gintonin-Mediated Neuroprotective and Injury-Preventive Effects by LPAR Antagonist Pretreatment in Human Brain-Derived Vascular Endothelial Cells (HBMEC)

After pretreatment with a nitric oxide synthase (NOS) inhibitor, the inventors of the present invention sought to determine whether the neuroprotective and damage-prevention effects of gintonin 10 ug/mL treatment were due to the functional ingredient lysophosphatidic acid receptor (LPAR) of gintonin.

Specifically, human brain-derived vascular endothelial cells imHBMEC (immortalized Human Brain Microvascular Endothelial Cell) were cultured in an appropriate culture medium under air conditions of 37° C., 5% carbon dioxide and 95% relative humidity, and when the cells reached 80 to 90% confluency. (average ~2 days), first of all, the cells were pre-cultured with an LPAR antagonist (Ki16425, 5 uM) for 30 minutes, then treated with 10 ug/ml of gintonin as in <Example 3> above, followed by incubating for 1 hour, and then, after treating with 100 uM of a synthetase inhibitor and incubating for 1 more hour, the effect on nitric oxide (NO) production was investigated.

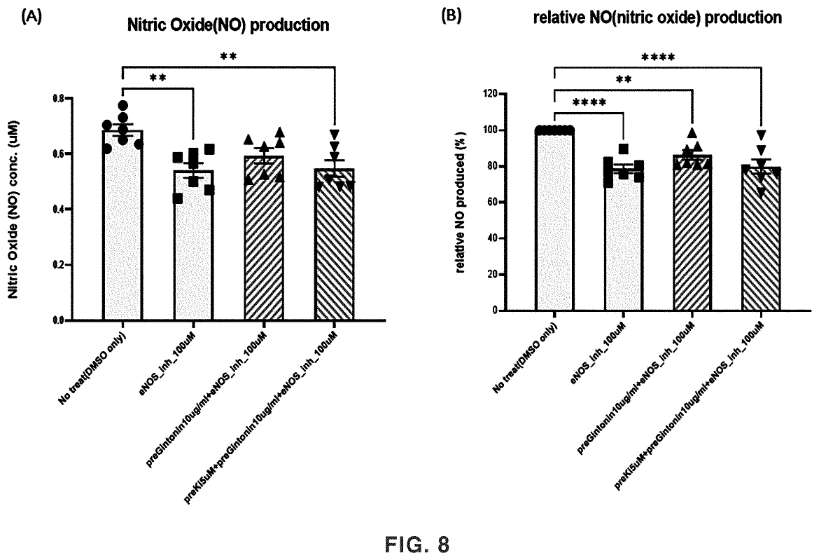

As a result, the nitrogen level reduced by the nitric oxide synthase inhibitor treatment was not recovered by gintonin when pretreated with the LPAR antagonist (Ki16425, 5 uM) for 30 minutes, indicating that the effect of gintonin is through LPAR inhibition (FIG. 8). FIG. 8A shows the results of analyzing how the production of nitric oxide (no treat (DMSO only)) produced by human brain-derived endothelial cells (imHBMEC) according to treatment conditions, the reduction in nitric oxide (eNOS_inh_100 uM) by the synthase inhibitor treatment (100 uM), and the change in nitric oxide production by gintonin treatment (preGintonin 10 ug/mL+eNOS_inh_100 uM) are affected by 30-minute pretreatment (preKi5uM+preGintonin 10 ug/ml+eNOS_inh_100 uM) with an LPAR antagonist (Ki16425, 5 uM). FIG. 8B shows the results of analyzing the relative nitric oxide (NO) production by treatment conditions.

As shown in the results of FIG. 8, since the reduced nitric oxide level after treatment with a synthase inhibitor (100, 300 uM) was not recovered by pretreatment with an LPAR antagonist (Ki16425, 5 uM) or by 10 ug/mL of gintonin, it can be inferred that the effect of gintonin was through LPAR inhibition. In addition, the results of ANOVA statistical analysis showed that FIG. 8A had a high significance with a p value of 0.0023, and FIG. 8B, which analyzed the relative nitric oxide production, showed a high significance with a p value of 0.0001.

### INDUSTRIAL APPLICABILITY

Gintonin, which includes an LPAR ligand-lysophosphatidic acid receptor (LPAR) derived from ginseng according to the present invention, increased the post-stroke survival rate and reduced the infarct volume of mice in a mouse model of focal ischemic stroke. Additionally, in cell experiments based on human brain microvascular endothelial cells (HBMEC), it was confirmed that the decreased nitric oxide (NO) level was restored by treatment with an inhibitor (NOSi) of nitric oxide synthase (NOS). Therefore, gintonin has the effects of being advantageously used for the prevention and treatment of an ischemic cerebrovascular disease, including cerebral infarction. Since it is expected to provide market competitiveness by providing the effect of reducing medical costs for treatment and the like, it has industrial applicability.

1. A method for treating an ischemic cerebrovascular disease, comprising administering a composition comprising gintonin to a subject in need thereof.

2. The method of claim 1, wherein the gintonin is lysophosphatidic acids in gintonin.

3. The method of claim 1, wherein the ischemic cerebrovascular disease is ischemic cerebral infarction.

4. The method of claim 1, wherein the ischemic cerebrovascular disease is caused by decreased nitric oxide production.

5. The method of claim 1, wherein the pharmaceutical composition reduces a volume of cerebral infarction.

6. The method claim 1, wherein the pharmaceutical composition increases a survival rate of a subject with cerebral infarction.

7. A method for protecting a nerve cell, comprising administering a composition comprising gintonin to a subject with an ischemic cerebrovascular disease.

8. The method of claim 7, wherein the gintonin is lysophosphatidic acids in gintonin.

9. The method of claim 7, wherein the nerve cell is a nerve cell that is damaged due to decreased nitric oxide production.

10. The method of claim 7, wherein the nerve cell is a nerve cell that is derived from cerebral blood vessels.

11. A health functional food composition for ameliorating an ischemic cerebrovascular disease and protecting a nerve cell, comprising gintonin.

12.-15 (canceled)
